(19) United States
(12) Patent Application Publication
(10) Pub. No.: US 2021/0299371 A1
(43) Pub. Date: Sep. 30, 2021

(54) AIR FILTRATION AND USER MOVEMENT MONITORING DEVICES

(71) Applicant: Good Interfaces, Inc., New York, NY (US)

(72) Inventor: Justin Lange, New York, NY (US)

(21) Appl. No.: 17/217,010

(22) Filed: Mar. 30, 2021

Related U.S. Application Data
(60) Provisional application No. 63/002,528, filed on Mar. 31, 2020.

(51) Int. Cl. A61M 16/00; A61M 16/06

(52) U.S. Cl. CPC A61M 16/0069; A61M 16/0666; A61M 16/0003; A61M 2205/82; A61M 2205/582; A61M 2205/75; A61M 2205/581

(57) ABSTRACT

Air filtration and user movement monitoring devices, as well as uses thereof are provided herein. An example device includes a power supply; an automated air blower mechanism coupled to the power supply; a filtration medium; a sealed nasal cannula with air ports; sensors configured to measure breathing patterns of a user of the device; a set of one or more channels connecting the automated air blower mechanism to the filtration medium and the sealed nasal cannula; a memory configured to store program instructions; and a processor operatively coupled to the memory to execute the program instructions to: modulate power supplied from the power supply to the automated air blower mechanism based on the breathing patterns of the user; and modulate temporal operation parameters of the automated air blower mechanism based on the breathing patterns of the user.

### FIELD

The field relates generally to device technology, and more particularly to wearable device technology.

### BACKGROUND

Particulate respirators and filter masks are used in many environments that contain airborne particles hazardous to human health. For example, particulate masks are used by construction workers and medical workers to avoid breathing in unsafe particles and/or pathogens. In addition, surgical masks, respirators, and other filtering devices are often used by medical workers as well as the general populace to reduce the risk of the wearer transmitting a disease to others by way of microdroplets expelled when speaking, breathing, coughing, sneezing, etc.

However, despite such benefits, filter masks impede some aspects of normal human functioning and interaction, thereby commonly rendering compliance low across many environments and contexts. For example, conventional filter masks typically make it difficult for wearer to talk, breathe, drink, eat, etc. In addition, conventional filter masks obscure an essential area of the human face necessary to convey micro-expressions. Therefore, users will commonly temporarily remove such conventional masks, or decline to wear them altogether.

In addition, conventional filter masks are routinely ineffective in eliminating disease transmission because the wearer may, for example, introduce pathogens or other objectionable foreign particles by touching their eyes, nose, and/or mouths (for example, by scratching their nose underneath the mask). Such a wearer might then also touch objects or people without first decontaminating their hands.

Accordingly, there exists a need for a device that provides filtration of incoming air but is minimally obstructive of a user's face, monitors the user's actions and alerts the user if he or she is about to touch their face or have already done so, and provides a guided, motivation framework to condition the user's respiratory and overall physiological health.

### SUMMARY

Illustrative embodiments provide air filtration and user movement monitoring devices, as well as uses thereof. An exemplary device includes at least one power supply, at least one automated air blower mechanism coupled to the at least one power supply, at least one filtration medium, and at least one sealed nasal cannula with one or more air ports. The device also includes one or more sensors configured to measure one or more breathing patterns of a user of the device, and a set of one or more channels connecting the at least one automated air blower mechanism to the at least one filtration medium and the at least one sealed nasal cannula. Further, the device includes a memory configured to store program instructions, and a processor operatively coupled to the memory to execute the program instructions to: modulate power supplied from the at least one power supply to the at least one automated air blower mechanism based at least in part on at least one of the one or more breathing patterns of the user, and modulate one or more temporal operation parameters of the at least one automated air blower mechanism based at least in part on at least one of the one or more breathing patterns of the user.

In another embodiment, an example method includes generating filtered air for a user of a mask device, wherein generating the filtered air comprises using at least one power supply, at least one automated air blower mechanism, and at least one filtration medium of the mask device, and measuring, using one or more sensors, one or more breathing patterns of the user inhaling at least portions of the filtered air generated by the device. The method also includes modulating power supplied from the at least one power supply of the mask device to the at least one automated air blower mechanism based at least in part on at least one of the one or more breathing patterns of the user, and modulating one or more temporal operation parameters of the at least one automated air blower mechanism based at least in part on at least one of the one or more breathing patterns of the user.

In yet another embodiment, an example method includes detecting, using one or more sensors embedded within a mask device worn by a user, at least one object within a given proximity of at least one of the one or more sensors and the mask device, and implementing, based at least in part on the at least one detected object, one or more outputs via one or more feedback components of the mask device.

Illustrative embodiments can provide significant advantages relative to conventional filter masks. These and other illustrative embodiments described herein include, without limitation, methods, apparatus, networks, systems and processor-readable storage media.

### BRIEF DESCRIPTION OF THE DRAWINGS

FIG. 1 is a block diagram of a mask device in an example embodiment;

FIG. 2 is a diagram illustrating a view of one or more components of a mask device in an example embodiment;

FIG. 3A and FIG. 3B are diagrams illustrating multiple views of one or more components of a mask device in an example embodiment;

FIG. 4 is a diagram illustrating a view of one or more components of a mask device in an example embodiment;

FIG. 5 is a diagram illustrating a view of one or more components of a mask device in an example embodiment;

FIG. 6 is a diagram illustrating a view of one or more components of a mask device in an example embodiment;

FIG. 7 is a diagram illustrating a view of one or more components of a mask device in an example embodiment;

FIG. 8 is a diagram illustrating a view of one or more components of a mask device in an example embodiment;

FIG. 9 is a diagram illustrating a view of one or more components of a mask device in an example embodiment;

FIG. 10 is a diagram illustrating a view of one or more components of a mask device in an example embodiment;

FIG. 11 is a diagram illustrating a view of one or more components of a mask device in an example embodiment;

FIG. 12 is a diagram illustrating a view of one or more components of a mask device in an example embodiment;

FIG. 13 is a diagram illustrating multiple views of one or more components of a mask device in an example embodiment;

FIG. 14 is a flow diagram illustrating techniques according to an example embodiment; and

FIG. 15 is a flow diagram illustrating techniques according to an example embodiment.

### DETAILED DESCRIPTION

As detailed herein, one or more embodiments include air filtration and user movement monitoring devices, as well as uses thereof.

Example and/or illustrative embodiments will be described herein with reference to exemplary mask devices or other types of processing devices. It is to be appreciated, however, that the invention is not restricted to use with the particular illustrative network and/or device configurations shown. By way of example, the term “network” as used herein is intended to be broadly construed, so as to encompass, for example, any system comprising multiple networked processing devices.

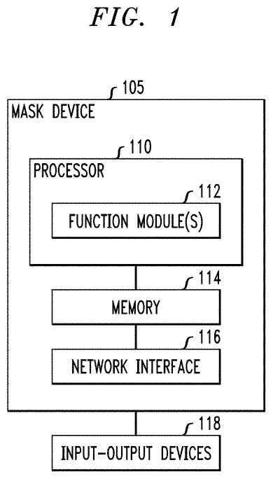

FIG. 1 is a block diagram of a mask device in an example embodiment. In at least one embodiment, the mask device 105 in the FIG. 1 embodiment can be implemented using at least one processing device. Each such processing device can include at least one processor 110 and at least one associated memory 114, and can implement one or more functional software modules 112 or components for controlling certain features of the mask device.

In the example embodiment illustrated in FIG. 1, the mask device 105 includes a processor 110 coupled to a memory 114 and a network interface 116.

The processor 110 can include, for example, a microprocessor, a microcontroller, an application-specific integrated circuit, a field-programmable gate array or other type of processing circuitry, as well as portions or combinations of such circuitry elements.

The memory 114 can include, for example, random access memory (RAM), read-only memory (ROM) or other types of memory, in any combination. The memory 114 and other memories disclosed herein can also be viewed as examples of processor-readable storage media, which can store executable computer program code and/or other types of software programs.

Examples of such processor-readable storage media can include, by way of example, a storage device such as a storage disk, a storage array or an integrated circuit containing memory, as well as one or more other types of computer program products. The term “processor-readable storage media” as used herein should be understood to exclude transitory, propagating signals.

The network interface 116 allows the mask device 105 to communicate over a network with other devices, and can include, for example, one or more conventional transceivers.

Additionally, the mask device 105 can be coupled to one or more additional devices such as mobile telephones, laptop computers, tablet computers, desktop computers or other types of computing devices. The mask device 105, in one or more embodiments, can also be coupled to respective computers associated with a particular organization or other enterprise. Numerous other operating scenarios involving a wide variety of different types and arrangements of processing devices and networks are possible, as will be appreciated by those skilled in the art.

Also, it is to be appreciated that the term “user” herein is intended to be broadly construed so as to encompass, for example, human, hardware, software or firmware entities, as well as various combinations of such entities.

Also associated with the mask device 105 are input-output devices 118, which can include, by way merely of example, keyboards, displays or other types of input-output devices in any combination. Such input-output devices 118 can be used to support one or more user interfaces (UIs) to the mask device 105, as well as to support communication between the mask device 105 and other related systems and devices not explicitly illustrated in FIG. 1.

Referring again to the depiction of the mask device 105, and as further detailed herein, the processor 110 can also include one or more function module(s) 112.

It is to be appreciated that this particular arrangement of modules illustrated in the processor of the FIG. 1 embodiment is presented by way of example only, and alternative arrangements can be used in one or more other embodiments. For example, the functionality associated with the modules in other embodiments can be combined into a single module, or separated across a number of modules. By way of further example, multiple distinct processors can be used to implement different ones of the modules, or portions thereof.

Also, at least portions of the modules in the FIG. 1 embodiment can be implemented at least in part in the form of software that is stored in memory and executed by processor.

It is to be understood that the particular set of elements shown in FIG. 1 is depicted by way of illustrative example only, and in one or more other embodiments, additional or alternative elements may be used.

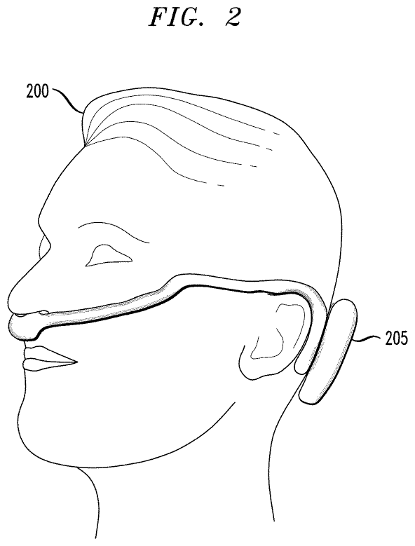

As further detailed herein, FIG. 2 through FIG. 12 are diagrams illustrating views of one or more components of a mask device in one or more example embodiments. For example, FIG. 2 is a diagram illustrating a view of one or more components of a mask device 205 being worn by a user 200 in an example embodiment.

Also, as noted herein, at least one embodiment includes air filtration and user movement monitoring systems, as well as uses thereof. Such an embodiment includes a lightweight, powered-air respirator that interfaces with the user's respiratory system via a sealing nasal cannula, with sensors to allow an embedded smart control system to correct undesired user behavior, such as accidental inspiratory mouth breaths as well as accidental and/or inadvertent face touching.

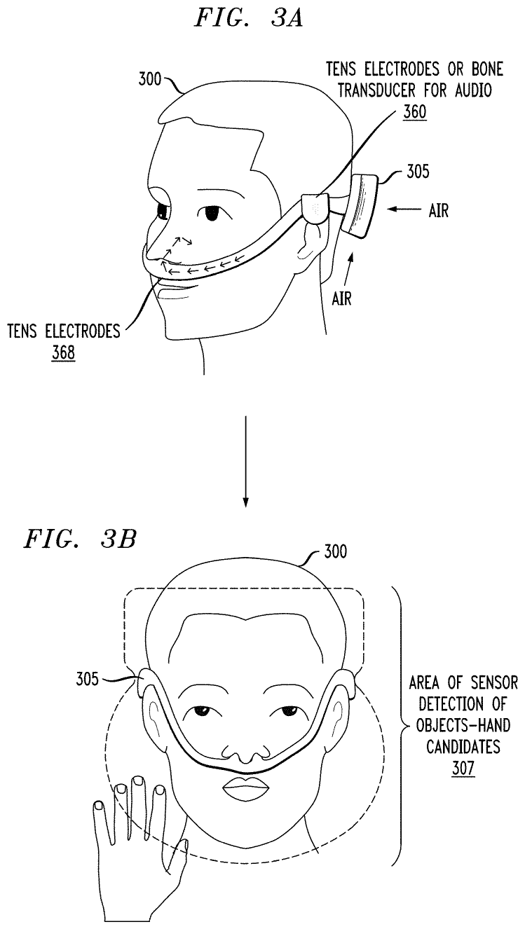

FIG. 3A and FIG. 3B are diagrams illustrating multiple views of one or more components of a mask device in an example embodiment. By way of illustration, FIG. 3A depicts a user 300 wearing a mask device 305 which includes electrodes (e.g., a set of one or more transcutaneous electrical nerve stimulation (TENS) electrodes) and/or a bone transducer (e.g., for audio) 360 positioned on the mask device 305 in and/or around a portion of the mask device which would typically sit above the user's ear. Additionally, FIG. 3A depicts electrodes (e.g., TENS electrodes) 368 positioned on the mask device 305 in and/or around a portion of the mask device which would typically sit under the user's nose. Further, the arrows depicted in FIG. 3A indicate the direction of airflow moving through the mask device 305.

By way of illustration, FIG. 3B depicts user 300 wearing mask device 305, and also depicts an area 307 of sensor detection with respect to hand movement (e.g., movement of the user's hands) and/or object movement, as further described herein.

In many cases, mouth-breathing represents an involuntary, subconscious adaptation to reduced openness of the nasal airway. In other words, humans typically only breathe through our mouths when we are unable to inhale sufficient air through our nose. Accordingly, one or more embodiments include actively assisting inspiratory nasal breathing using at least one efficient electrical blower.

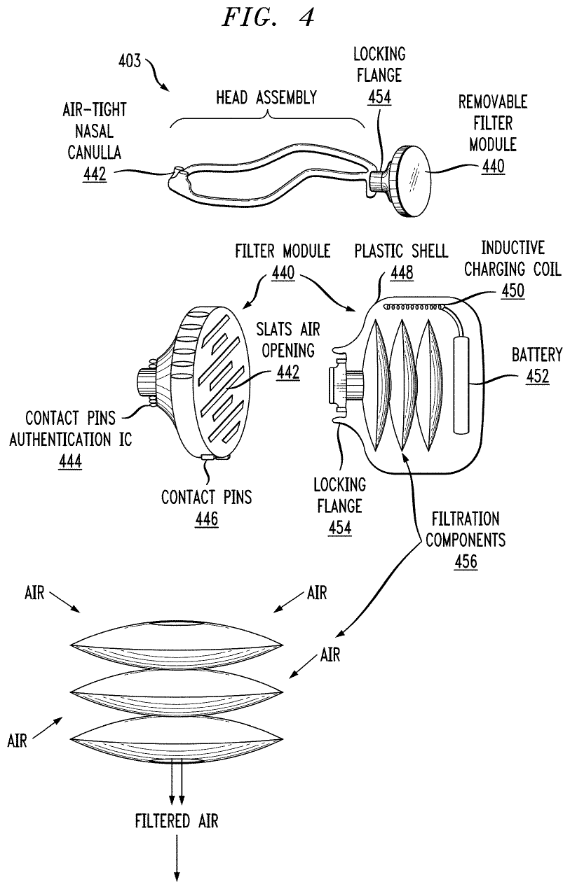

FIG. 4 is a diagram illustrating a view of one or more components of a mask device 405 in an example embodiment. In at least one such embodiment, a blower is housed within a removable, low-profile filter module 440 (encased with a shell 448 which includes air slot openings 442) that contains one or more particulate filtration components 456 (e.g., filter discs), a (rechargeable) battery 452, an inductive charging coil 450, and one or more sensors (detailed further herein). The lightweight assembly attaches to a locking flange 454 positioned on the mask device typically associated with a position behind the user's head. The locking flange 454 connects to a head assembly that closely follows the contour of the user's head, with unobtrusive (clear) plastic tubes that connect to a nasal cannula 442 that seals underneath the user's nose. When the user breathes in, filtered air is drawn in through the filter module, assisted by the blower, and travels through channels (e.g., tubes) in the head assembly into the nasal cannula, and then into the user's lungs. The removable filter module 440 can be swapped with other filter modules, which can be charged in a portable carrying case.

Additionally or alternatively, in at least one embodiment, one or more portions of the mask device is configured as one module. For example, in such an embodiment, the battery 452 and the filter module cartridge 440 are configured as one piece and can be removed together and charged (and/or changed or sterilized) separately. Accordingly, in such an embodiment, contact pins 444 and contact pins 446 mechanically enables power and data to pass between the filter module 440 (which can include battery 452) and portions of the head assembly 403 of the mask device. Both contact pins 444 and 446 authenticate the mask device to ensure that the mask device is authentically manufactured as part of the protection for the user, and also record the remaining lifespan of the filtration component(s) 456 and/or the filter module 440 (or sub-module(s) thereof). In at least one example embodiment, whenever the filter module 440 module is put on a charger or attached to the mask device, information can be read from the filter module 440 and/or updated in connection with the filter module 440.

Also, one or more embodiments can include at least one light guide for optical sensing (including, for example, including ultraviolet (UV) light, visible light, and infrared (IR) light) implemented within one or more of the channels (e.g., tubes). For instance, in at least one embodiment, range-finding and thermal sensors mounted on the head assembly can detect the close proximity of objects such as human hands.

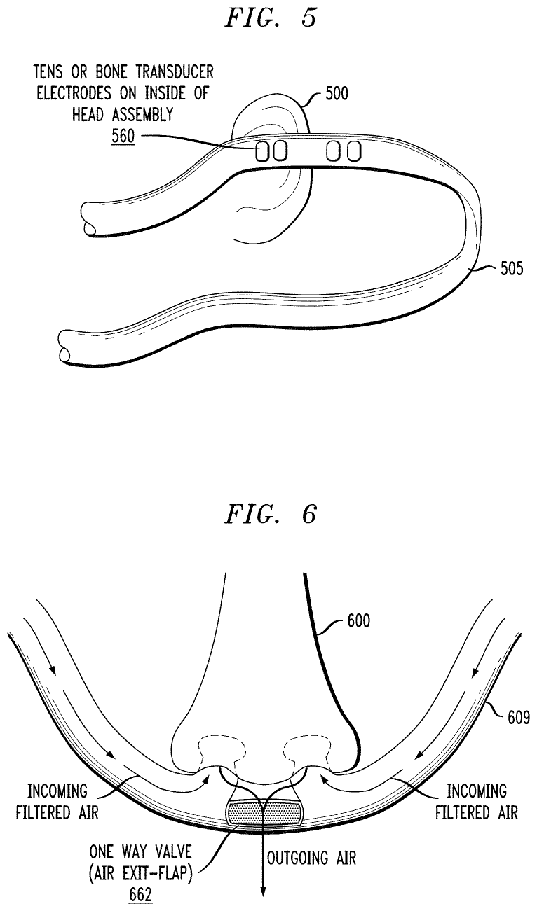

FIG. 5 is a diagram illustrating a view of one or more components of a mask device in an example embodiment. By way of illustration, FIG. 5 depicts a mask device 505 (worn by user 500) which contains electrodes (e.g., TENS electrodes) and/or a bone transducer 560. A bone-transducing audio output within the head assembly allows the device to transmit audio to the user without earbuds. For example, when the user moves his or her hands within a few inches of their face, an immediate audio trigger (audible to the user) alerts the user in time for him or her to reverse and/or stop their action. Electrical stimulation electrodes (e.g., electrodes 560) and/or vibration motors allow the user to optionally configure the device to provide a feedback that is effective for behavioral conditioning. In one or more embodiments, both audio and electrical simulation outputs are combined and/or utilized with other haptic feedback such as, for example, vibration.

In at least one embodiment, the device does not need a companion software application to function, but a companion smartphone application can be implemented to extend the functionality of the device in one or more meaningful ways. For example, such an application can allow the user to take part in respiratory exercises and track their respiratory fitness. It can also allow the user to engage with mindful breathing exercises, and track his or her progress.

FIG. 6 is a diagram illustrating a view of one or more components of a mask device in an example embodiment. By way of illustration, FIG. 6 depicts a channel 609 of a mask device (worn by user 600) includes and/or connects with a one-way valve 662 (also referred to herein as an air exit flap). FIG. 6 also depicts the directionality of incoming filtered air (e.g., filtered by one or more separate components of the mask device 600) through channel 609 towards the nose/nostrils of the user 600, who then breathes out through his or her nose/nostrils through the one-way valve 662.

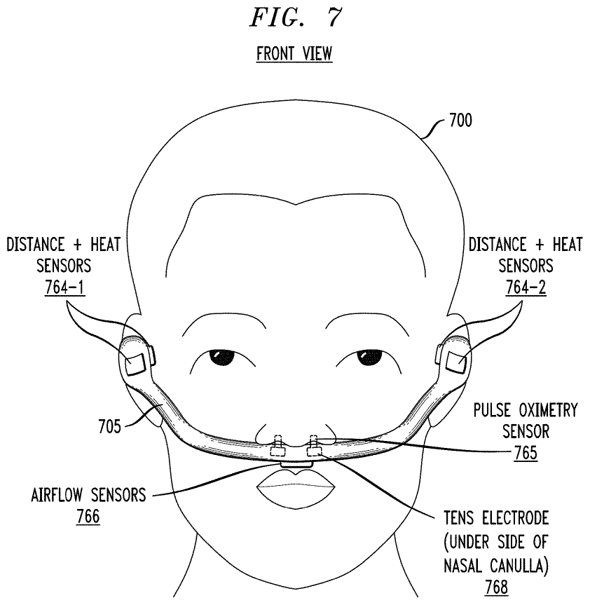

FIG. 7 is a diagram illustrating a view of one or more components of a mask device 705 in an example embodiment. As depicted in the FIG. 7 example, at least one embodiment can include detecting, via the device 705, breath drawn in (by user 700), and in response, turning-on at least one air blower only when breath is drawn in. The speed of the air blower can be increased proportional to the intensity of the breath being drawn in. Such a feature can save power, as the blower is powered off when the user is blowing out (exhaling). In some embodiments, the blower can optionally harvest power when the user is blowing out (e.g., in a challenge mode).

The device can detect whether user is breathing in through the nose or mouth, and remind the user to breathe through his or her nose. Specifically, the device 705, via airflow sensors 766 (e.g., distance and/or heat sensors), for example, detects the user breathing-in through nose or through the mouth, triggering at least one feedback, via electrodes (e.g., TENS electrodes) 768, for example, if the user breaths-in through the mouth. Such feedback conditions the user to be dominantly a nasal breather. Also, in one or more embodiments, the device can include a sticky pad (positioned, for example, on the user's chest) with at least one transducer on or near the user's neck, wherein the transducer wirelessly transmits a sensor steam and/or is connected via a wired connection. The stream may include data from a transducer, light sensor, etc. Additionally or alternatively to the above-noted sticky pad, such an embodiment can include a wearable device that affixes, for example, to the inside of the user's clothing, and/or a strap similar, for example, to a heart rate monitor.

Also, in one or more embodiments, the device 705, via airflow sensors 766, distance and/or heat sensors 764-1 and/or 764-2, for example, detects if user brings his or her hands close to his or her face, triggering a reinforcing feedback, via electrodes (e.g., TENS electrodes) 768, for example, to decondition such user behavior. Additionally, in at least one embodiment, the device detects when a user is about to sneeze and induces the user to abort the sneeze. Such an embodiment includes using an electroencephalogram (EEG), audio output, and artificial intelligence (AI) techniques to classify the incoming sensor data as predicting that the user is about to sneeze, and a program to induce the user to abort the sneeze by behavioral deconditioning via transcutaneous electrical nerve stimulation, for example (via electrodes 768), on the upper lip of the user.

At least one embodiment also includes monitoring and/or tracking one or more cardiovascular factors (heart rate, oxygen) of the user, for example, using pulse oximetry sensor 765, as well as conditioning the user's physiological health. Such an embodiment can include implementing physiological challenges with respect to the user (with gamification) to meet certain expiatory targets, such as volume exhaled and inhaled over time. Such challenges can be carried out one or more times a day, for example, and the user's progress is tracked. Accordingly, the user's respiratory capacity and overall health can be increased, for example, through training via an alternate nostril breathing regimen. Such an embodiment includes using a nasal cycle neuro-immunological conditioning engine to detect alternate nostril breathing by the user, and learn the user's respiratory patterns over time. Additionally, in one or more embodiments, the device can favor the direction of air to one side or the other (of the user's nose) to stimulate health respiratory function. Such training can also include breath retention challenges, which can, for example, prompt the user to retain breath for as long as possible and/or prompt the user to carry out one or more additional breathing training techniques. In conjunction with such embodiments, a companion software application can be generated and/or implemented to facilitate gamifying, prompting, and/or incentivizing the user to regularly engage in conditioning exercises. The targets and/or results of such exercises can, for example, be set and measured in such a companion application.

As also detailed herein, in one or more embodiments, the device includes at least one air cartridge that is pleated to increase surface area of a filter medium, a one or more bone conduction features. Such an embodiment can also include a mechanism to authenticate filter modules, whereby a security-based integrated circuit (IC) in the filter module(s) securely communicates with the mask device. The mask device can refuse to operate, for example, if a non-genuine filter cartridge is in place.

In one or more embodiments, the device can also include a search engine component, one or more audio output (e.g., earbud) components, and gestural override to allow the user to touch his or her face (for example, before a timeout). At least one embodiment can additionally include implementing a carrying case, in conjunction or in association with the mask device, for filter one or more cartridges (e.g., one cartridge can be charging in the case while another cartridge is in service in the mask device). Such an embodiment can also include a sanitizing case, which can include space for positioning the mask on a cradle (in the case), and means for closing the case and recharging the mask while using UVC light to disinfect the mask device (including the inside of the air channels (e.g., tubes)).

Further, at least one embodiment can include a fiberoptic or other light guide that inserts into the inside of the cartridge and/or other parts of the mask device to disinfect portions of the device. One or more embodiments can also include an ozone mode, which enables a case, if left outside for a given amount of time, to generate (e.g., via a certain bulb) ozone gas which can move through portions of the device to disinfect the device.

Additionally, at least one embodiment includes, via the device, collecting data on environmental interactions. For example, such an embodiment can include measuring, via distance and/or heat sensors 764-1 and 764-2, for example, the number of objects that a user touches, the user's capacitance changes over time, as well as the user's proximity to other users (e.g., the sustained heat presence of IR (measured via an IR sensor) indicates presence of a human).

As detailed herein, one or more embodiments include enabling and/or providing inspiratory breath augmentation. For example, such an embodiment includes implementing a mask device to function similarly to a powered air-purifying respirator that is lightweight and head worn. An example mask device includes a filtration medium, a power supply, and an air blower. Additionally, such a mask device detects when a user begins to breathe-in nasally, and then engages the air blower to increase the airflow available to the user for as long as the user is breathing-in. Such an increase in airflow can be, for example, proportional to the effort of the user, such that an easeful, comfortable breathing experience may be maintained regardless of how intensely the user may be breathing.

Accordingly, at least one embodiment includes mapping airflow assistance to respiratory effort. In such an embodiment, a software controller modulates the intensity of a powered air-purifying respirator (PAPR) breathing assistance mechanism such that the user's perceived exertion is ideally uniform, or ideally easeful, no matter how hard the user is breathing. Several modes of mapping the amount of device augmentation to inspiratory-respiratory exertion are available to the user, some of them user-configurable, and may be selected in a companion software application, via an interface on the mask device, or via a companion device (e.g., a remote controller, such as a watch, pendant or other wearable or portable item containing a transceiver). Such inspiratory-breath-augmentation modes may include modes wherein the user generally provides more effort to breathe relative to non-filtered breathing, modes wherein the user generally breathes normally such that their perceived exertion is no more nor less than their general non-filtered breathing, and modes wherein the user is able to breathe-in more easily than their general non-filtered breathing.

Additionally, one or more embodiments include mapping airflow assistance to physiological parameters. In such embodiments, inspiratory-augmentation mode parameters are adjusted based on other device inputs, such as the calculated overall metabolic exertion of the user, the remaining battery life of the mask device, the user's current oxygen saturation level, the user's heart rate relative to their calculated or known maximum heart rate, the user's heart rate variability, or a combination of any of these factors. The combination of one or more of these factors constitutes an adaptive inspiratory augmentation engine. The various factors and parameters selected by the user, as well as their data, may be periodically uploaded to a cloud service wherein the model may be changed or enhanced via machine-learning processes, and subsequently disseminated to the individual user devices such that the heuristics and reliability of this engine may be improved.

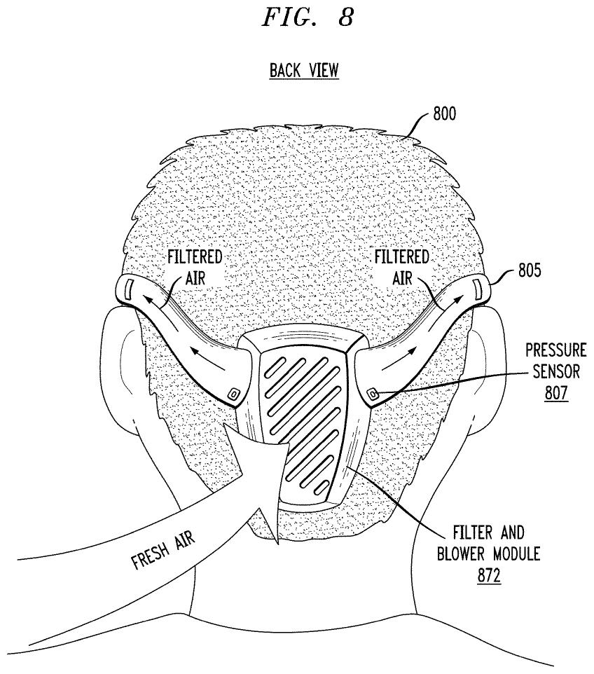

FIG. 8 is a diagram illustrating a view of one or more components of a mask device in an example embodiment. By way of illustration, FIG. 8 depicts a mask device 805 (worn by user 800) which includes a filter and blower module 872 and at least one pressure sensor 870. Also, as detailed herein, a mask device (e.g., mask device 805) implemented in connection with one or more embodiments can detect inspiratory nasal breath in a number of ways. For example, such detection can be carried out via at least one transducer. In some embodiments, a transducer in the inspiratory path converts the movement of air into a voltage that is proportional to the amount of air movement. The device then activates the blower mechanism 872 based at least in part on that conversion. Detection of respirator effort can also be carried out, for example, by measuring blower fan speed. In some embodiments, the typical speed of the air blower is known to the device (for any given power typically supplied to the air blower). Because sufficiently effortful inspiratory breath decreases the pressure within the device downstream of the filter component, pulling air into the filter beyond the volume of air that the blower would ordinarily move, a slight increase in speed of the revolutions per minute (RPM) of the blower relative to the expected RPM allows the controller to infer that the user is having to exert above-normal effort breathing. The controller then appropriately increases the RPM of the blower. As used herein, it is noted that “blower” refers to one or more blower(s), fans, compressors or other mechanical or micromechanical devices (e.g., module 872) that may, via digital or analog control, decrease, increase or stop the airflow within a surrounding area, volume or assembly. Accordingly, one or more embodiments include measuring the speed of the fan. It is known how fast the fan typically moves for any given power input, and if a deviation from that expectation is measured, it is determined that the user is helping move the fan along, and the power input can be adjusted accordingly.

In one or more embodiments, as also depicted in FIG. 8, detection of respirator effort can also be carried out using one or more pressure sensors 870. In such embodiments, at least one pressure sensor 870 installed within the inspiratory pathway (i.e., somewhere within the internal volume of the device between the inner side of the filtration medium and the user's nasal cavity) sends a digital signal or otherwise changes its output (e.g., voltage) in response to changes in pressure. At least one pressure sensor 870 installed elsewhere allows the device to determine outside atmospheric pressure. Based on the difference between these pressure readings, the device 805 modulates the blower speed.

At least one embodiment additionally includes harvesting power from a blower of the mask device. The blower is nominally off when the user breaths out. However, in some embodiments, the device may harvest power from expiratory breath by directing the airflow back through the blower mechanism and generating electricity from the rotation of the blower blades relative to motor magnets.

Also, one or more embodiments can include determining expiratory respiratory effort. In such embodiments, one or more valve) within the nasal pillow can be opened or closed by the device, such that out-breaths are fed back through one or more channels (e.g., tubes) within the head assembly. Sensors and processes, such as those described herein for determining inspiratory effort, can be utilized by the device to calculate expiratory effort.

One or more embodiments also include instrumental conditioning of nasal breathing. Because at least one embodiment does not provide for filtration of air that enters through the user's mouth, it is important that the user usually breath-in via their nose. However, an occasional inspiratory mouth breath does not defeat the usefulness of such an embodiment.

The device, in one or more embodiments, detects mouth-breathing in a number of ways. For example, a transducer embedded in the device can make contact with the skin (e.g., the trachea, face, temples, back of neck, etc.), and the movement of inspiratory and expiratory breath vibrates human tissue, creating a distinct sonic signature. Such a sensor can be referred to herein as a respiratory-measuring transducer. Also, one or more additional transducers embedded elsewhere within the device may preferentially measure ambient sound (e.g., sounds not originating from the body), such that the actual sonic signature coming from the user's body may be differentially obtained by comparing these two sensor readings. By way of example, if the user is speaking, the respiratory-measuring transducer will register a substantial signal, but the ambient-measuring transducer will register a substantial signal as well, which allows the device to determine that the an inspiratory breath is not taking place, as an inspiratory breath will not register a substantial signal from ambient measuring sensor(s). As described herein, the device may reliably detect inspiratory nasal breath. By comparing the determination of inspiratory nasal breath with the determination of inspiratory breath overall, an inspiratory breath detected may be characterized as a mouth breath if the concurrent detection of a nasal breath does not occur.

In some embodiments, at least one of the measuring sonic transducers are located within the ear canal or elsewhere on the ear, embedded in wireless or wired earbuds. Because opening one's mouth dramatically alters the acoustic resonance of the throat and mouth, mouth and nasal breathing typically sounds different. Analysis of the spectral characteristics by one or more embodiments allows breath to be classified as nasal or mouth.

At least one embodiment also includes digital signal processing. In such an embodiment, the signal from the respiratory-measuring transducer(s) and other sensors may be characterized via one or more signal processing techniques, such as a fast Fourier transform (FFT) and/or one or more machine learning techniques, such that not only is the amplitude of the device's variously placed acoustic transducers differentially considered, but the frequency content of the signal is analyzed within the device and compared to the signal characteristics of known respiratory events.

Additionally or alternatively, in one or more embodiments, at least one thermistor is embedded within the device, such that breathing out causes a change in the voltage of the sensor which can be used to determine the presence of warm expelled air. In other example embodiments, an accelerometer and/or gyroscope can be implemented on the user's chest and on the mask device, whereby such an embodiment includes subtracting head movement and taking the difference to determine breath patterns. Another example embodiment includes using one or more forms of radar to measure whether the user's mouth is open or not. Further, other example embodiments can include using a Peltier element, thermistor, passive IR sensor, and/or other temperature sensor in a nasal pillow (e.g., one sensor pointing down (towards the mouth), one sensor pointing up, and determining that whichever sensor is hotter (after calibration to equilibrium) is the orifice breathing outward).

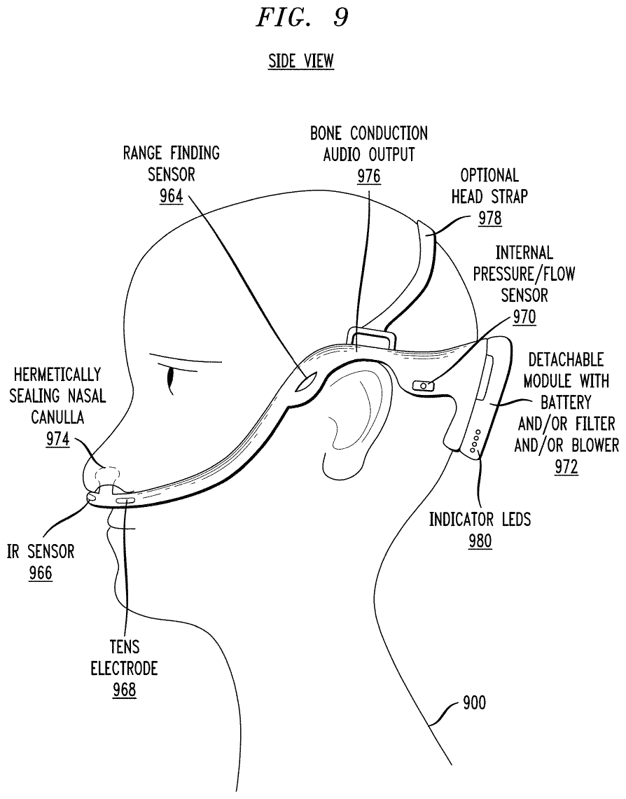

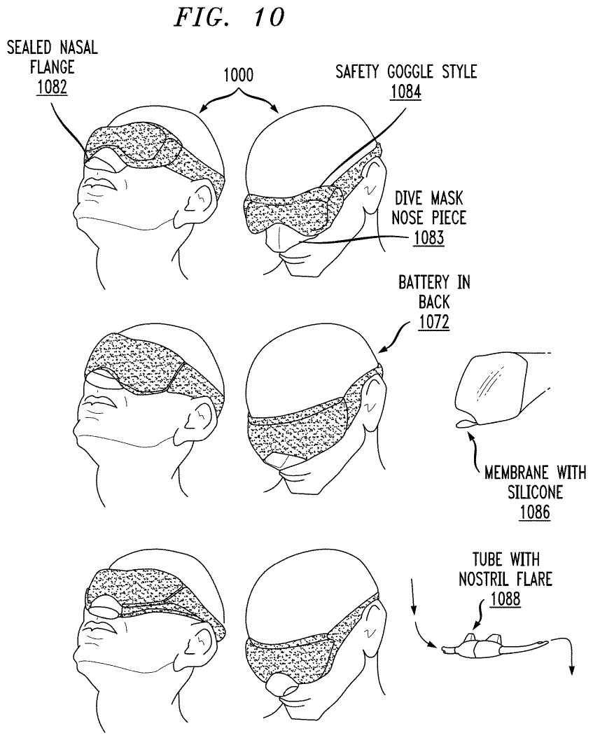

FIG. 9 is a diagram illustrating a side view of one or more components of a mask device in an example embodiment. By way of illustration, FIG. 9 depicts a mask device 905 (worn by user 900) which includes one or more IR sensors 966 in a nasal pillow along with one or more electrodes (e.g., TENS electrodes) 968, and a hermetically sealed nasal cannula 974 proximate thereto. As also depicted in FIG. 9, mask device 905 includes at least one range-finding sensor 964, a bone conducting audio output generator 976 (e.g., bone transducer), an optional head strap 978, one or more internal pressure and/or flow sensors 970, one or more indicator lights (e.g., LEDs) 980, and a detachable module 972 which includes a battery and/or a filter and/or a blower. In one or more embodiments, LEDs 980 indicate the remaining life of the battery and/or indicate the remaining life of the filter cartridge (within module 972). Additionally or alternatively, at least one display (e.g., an organic LED (OLED) display) can be implemented in place of or in addition to LEDs 980 to display some or all of the above-noted information (e.g., “remaining filter life: 23 days, 4 hours,” “remaining battery life: 9 hours, and 30 minutes,” etc.).

As also depicted in FIG. 9, range-finding sensor 964 (e.g., an IR sensor, a radar sensor, etc.) detects the distance and position of objects, such as the user's hand(s), in conjunction with one or more other sensors such that the mask device can alert the user (e.g., if the user touches his or her face).

In at least one embodiment, the mask device induces initiating at least one physiological trigger (e.g., via electrodes 968) when mouth-breathing is detected. In such an embodiment, when the device detects the beginning of an inspiratory mouth breath (using, for example, IR sensor(s) 966), the device provides a sequence of feedback, which may include applying electrical stimulation (ES) such as transcutaneous electrical nerve stimulation to the user's face (via electrodes 968, for example). Other feedback mechanisms can include audio transducers, vibration motors, etc. In some embodiments, the intensity of the ES applied to the user's skin is proportional to the continuous time or amount of the detected inspiratory breath.

Also, one or more embodiments include training a breathing detection engine. In such an embodiment, the controlling software or firmware module making the determination of whether the user is breathing in through their mouth versus breathing in through their nose is at least partially operating through machine learning (ML) processes. A physical input modality (such as a capacitive pad, for example), a near-field non-tactile input modality (such as an IR or distance switch, for example), or other input modality (such as speech recognition, for example) allows the user to indicate if the detection of a mouth breath was incorrect, which provides the system with another data point to improve the accuracy of its determinations.

Further, one or more embodiments include suspending conditioning (e.g., pressing a button to pause reinforcement feedback). In such an embodiment, one or more input modalities (e.g., tactile, near-field/gestural, head-gestural, auditory or connected remote (i.e., application, dongle, etc.)) temporarily suspend the operant conditioning mode, such that for a period of time or one or more detected mouth breaths, the device will produce no reinforcement feedback (no TENS, haptic, etc.).

Also, in at least one embodiment, the low-latency detection and interruption of indeliberate, impending contact between a user's face and the user's hands is a feature of the device. A combination of sensors and processing is used to detect the close proximity of objects to the user's face and accurately classify such objects (e.g., classify such objects as hands). The device can, for example, detect hands when the hands are within a given proximity to the front hemisphere of the user's head, specifically when the hands are within an area in which it would be possible for fingers to touch the nose, mouth or eyes of the user. When a user is about to touch his or her face, the device can induce the user to stop such action. In such an embodiment, multiple types of sensing technologies can be employed. For example, one such sensing technology characterizes the distance and motion of the object(s), and another such sensing technology characterizes the emissive radiation (e.g., infrared radiation of the object(s)).

One or more embodiments additionally include distance and range sensing. For example, one or more digital ranging modules can produce a high-accuracy determination of the speed and relative distance of nearby objects. At high refresh rates, sub-millimeter (mm) accuracy can be enjoyed with relatively low-power consumption, provided that the detection range, for example, is not more than several feet away from the sensor.

Many distance sensing technologies, such as LIDAR, TOF, pulsed coherent radar (PCR), and ultrasonic sensing can be utilized by such embodiments. In an example embodiment utilizing PCR, not only is the distance of a detected object known but the speed is known as well, such that for a given hand or arm movement wherein the user is proceeding on a vector that will bring their hands in contact with their face, the probable time to physical contact may be calculated, which is then input to the device controller. The output of the one or more distance sensing modules can be processed in such a way as to determine not only the distance of the object(s) (e.g., hand(s)) from the sensor, but the distance of the object(s) from any area of interest within the front hemisphere of the face (e.g., the eyes, nose or mouth). This is accomplished by the device controller having a generic map of a human face in its memory.

Accordingly, at least one embodiment includes calibrating at least one face map. For example, during the setup of the device, the user can be prompted (via a connected software application, via accompanying earbuds, or via a device internal speaker) to touch different areas of their face with different hands and different fingers, moving at a constant speed until the finger touches the face, and then holding still until prompted. For instance, the user interface controller can play an audio recording, that states “Now extend the index finger of your right hand outward and point to the horizon, and then, moving slowly, touch this index finger to your left temple, reversing direction after you touch the eyebrow.” The delta between the device's generic face map and the user's touch points is then stored in memory, and a post-processing phase is carried out by the device wherein the points are interpolated within the existing generic dataset defining human facial structures (with or without machine learning, such that a touch point farther forward than the generic depth map does not merely create a bump in the new depth map but rather brings forward that feature, which is recontoured within the context of all neighboring features according to the definitions of the generic facial feature set) to produce a new depth map that is calibrated individually to and/or by the user.

In at least one embodiment, a range-finding module (e.g., sensor 964 in the example FIG. 9 embodiment) outputs to the device controller the coordinates, speed and vector of nearby objects relative to itself (e.g., candidates for classification as hands). The device controller, having in memory the precise location of the range-finding module(s) and a depth map of the user's face, may calculate the distance and angle between the rangefinder module(s) and any point on the user's face. Thus, the device may calculate the distance and vector of any incoming object relative to any point on the user's face. When objects come within a certain distance threshold of the user's face, the device controller iterates through a set of points representative of all relevant parts of all facial features, and compares these to the closest-most hand-candidate object, as well as the centroid or machine learning-derived anatomical anchor point for a hand-candidate object, such that the relative distance and immediate time-to-contact may be calculated not only for the closest-most point of the hand-candidate object, but also for the finger of a hand-candidate object if it were in the future to suddenly be extended, while at the time of this process observed or assumed to be unextended.

As noted herein, at least one embodiment can include utilizing infrared sensing. Such an embodiment uses at least one sensor that can convert mid-IR (e.g., the mid-IR energy radiated by humans) into electrical current. One or more sensors can provide a steady stream of accurate IR and temperature readings to the device controller. One or more custom-molded Fresnel lenses (e.g., made from HDPE or another IR-transmissive material) can refract IR energy from the areas surrounding the face, while occluding from the sensor view those areas not surrounding the face. Such lenses can be designed such that IR energy from areas not adjacent to the front hemisphere of the head are occluded from the sensor, such that, for example, touching the back of your neck does not trigger a positive hand-detected reading.

In one or more embodiments, even if incoming objects or nearby objects can be classified as hands, an infrared sensor can be used to substantially improve the accuracy of the classification. For example, if a user is getting into an automobile, their head will typically come within a few inches of the doorframe, which could cause a range-finding-only solution to inadvertently trigger a positive hand-detected reading. However, the doorframe of an automobile does not emit the IR radiation of the human body. Accordingly, in at least one embodiment, the device controller only classifies an incoming or nearby object as a human-hand and/or forearm if the one or more IR sensor(s) register its human-like heat signature.

Further, in one or more embodiments, the reflex speed of the user is calculated from the user's known age and/or via prompts through a game-like test (e.g., to tap a button in response to sensory stimuli) such that their normal reaction speed can be known. Because the distance of the user's hands (from their face) as well their speed can be known via range-finding sensor technologies such as PCR, and moderate changes in speed can be anticipated or assumed, the probable time to contact (the user's face) can be predicted. Accordingly, the system controller can wait to alert the user (until just before such contact occurs) by calculating the time that it will take the user to reverse their hand and/or arm motion, and optionally adding a small buffer of time to this calculated user reflex time.

When the system determines that the user is about to touch their face, a physiological trigger is induced. Such a trigger may include one or more of the following: EM such as TENS, sound played over speakers, sound in earbuds, vibration, light, etc.

In addition to the classification of breathing as inspiratory nasal breathing, expiratory nasal breathing, expiratory mouth breathing, or inspiratory mouth breathing, the device may, in at least one embodiment, classify other respiratory events including classifying the stage of respiratory events such that a usually-subsequent stage of that respiratory event may be anticipated. In some modes, if the device anticipates that a user is about to sneeze, the device induces a physiological trigger such that the user is encouraged to abort the completion of this reflex.

By way merely of illustration, one can appreciate that a sneeze can be considered as happening in several stages, and it should be noted that sneezes often occur in response to the sensation of foreign bodies in the nose and/or sinus system. Because the mask device of one or more embodiments filters out contaminants from the air before they enter the user's nose, the triggers which typically induce sneezing are already reduced. In the case that filtration does not entirely prevent the sneeze reflex from occurring, it is known that a strong unexpected physiological or emotional input to the user's experience can interrupt the sneeze process. Accordingly, in one or more embodiments, the respiratory event classification engine determines that the completion of a sneeze is imminent by comparing the intensity of the current detected inspiratory breath to a set of previous inspiratory breaths. Further, in such an embodiment, an electrode for this stimulation is located within the nasal pillow, such that stimulation is applied to the nose region. In some embodiments, a sequence of colored light can be output, sound can be played over speakers and/or in earbuds, and/or vibration can accompany the physiological stimulus.

Moreover, in one or more embodiments, the respiratory event classification engine determines that the completion of a sneeze is imminent by feeding some or all sensor data through a recurrent neural network (RNN) or other neural network such that an output with a classification probability in excess of a certain threshold is identified.

As also noted herein, at least one embodiment includes monitoring and/or tracking cardiovascular parameters. As described herein, the accuracy of the various behavioral classification engines of the device controller can benefit from additional inputs describing the user's physiological state. In some embodiments, the device contains one or more heart rate monitor modules constituting electrodes and/or optical sensors as well as a specific light-emitting diode (LED) emitter.

Additionally, pulse oximetry measures the oxygenation of blood by measuring light through cartilage. In one or more embodiments, a pulse oximetry module is housed in the nasal pillow module (i.e., the pulse oximetry is read through the septum of the user's nose). In one embodiment, a pulse oximetry module is situated in or around the ear, such that the coded light source shines a light through the proximal side of the helix of the external ear, which is read by sensors and/or electrodes on the wired or wireless earbuds situated in the concha auricular.

As noted herein, an additional function of at least one embodiment includes prompting the user with respiratory challenge exercises and/or meditations. Such an embodiment can include generating and/or implementing a companion software application, which allows the user to schedule and choose such exercises, as well as prompt the user when it is time to do them, and incentivize user to regularly engage in conditioning exercises. The respiratory targets and activities, as well as the results of such exercises, are set and measured in the companion application.

As also detailed herein, in at least one embodiment, filter modules contain the filter and a rechargeable battery. Such modules can be swapped with other modules, which can recharge in a portable charging case while not in use in the mask device. The charging case can have UVC sterilizing bulbs so that contamination is not an issue. Also, a smart IC (e.g., a security IC or another memory chip) within the filter module allows the device to track the number of hours that the filter is being used, and the device informs the user when a filter is close to the end of its life.

One or more embodiments can also include authenticating filter modules. For example, a security IC in the filter modules securely communicates with the mask device, such that the mask can refuse to operate if a non-genuine filter cartridge is in place. This communication can take place via contact pins or contact blades, or via wireless communication protocols such as RFID or NFC.

At least one embodiment can also include measuring (permissible) exposure levels to one or more agents. For example, such an embodiment includes implementing an on-board particulate sensor (and/or sensors for other agents, gases, etc.), which measures permissible exposure levels and accidental mouth-breaths, and alerts the user if the user is approaching any upper limits. By way of illustration, total user exposure=(% of contaminant in air*mouth breathing)+(% of contaminant in air*nasal breathing/filtration medium efficacy).

In one or more embodiments, an additional module houses multiple transducers and close couponing housing for the transducers such that the transducers are approximately flush with the user's back and chest in a variety of different locations, corresponding to the anatomy of the lung.

FIG. 10 is a diagram illustrating a view of one or more components of a mask device in an example embodiment By way of illustration, FIG. 10 depicts an example embodiment which includes coupling the mask device (worn by user 1000) with goggles or other protective eyewear 1084. As further depicted in FIG. 10, such an embodiment can also include a sealed nasal flange 1082 as part of a mask nose-piece 1083 with a membrane covering 1086 (e.g., a silicone membrane), as well as a battery 1072. Additionally or alternatively, as also illustrated in FIG. 10, such an embodiment can include a channel (e.g., a tube) with one or more nostril flares 1088.

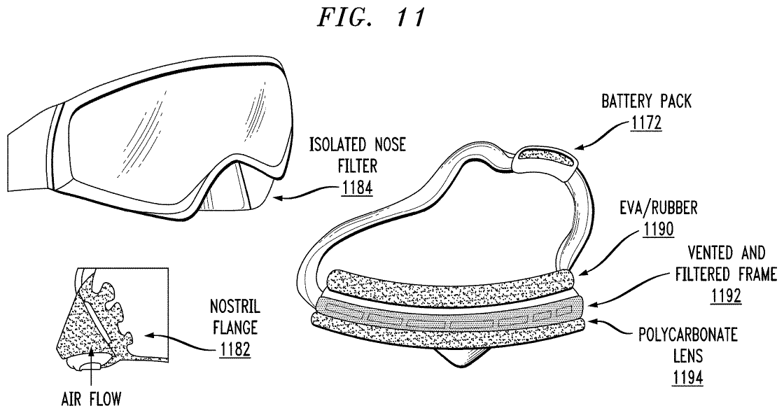

FIG. 11 is a diagram illustrating a view of one or more components of a mask device in an example embodiment. Similar to the embodiment depicted in FIG. 10, FIG. 11 depicts a mask device which includes eyewear and nose-piece components such as isolated nose filter 1184, nostril flange 1182, polycarbonate lens 1194, vented eyewear frame 1192, rubber gasket 1190, and battery pack 1172.

As illustrated in FIGS. 10 and 11, such example embodiments include implementing and/or incorporating safety eyewear with the smart mask device functionality detailed herein. In such an embodiment, the mask device could, for example, be implemented to cover a user's nose and eyes, but not the user's mouth.

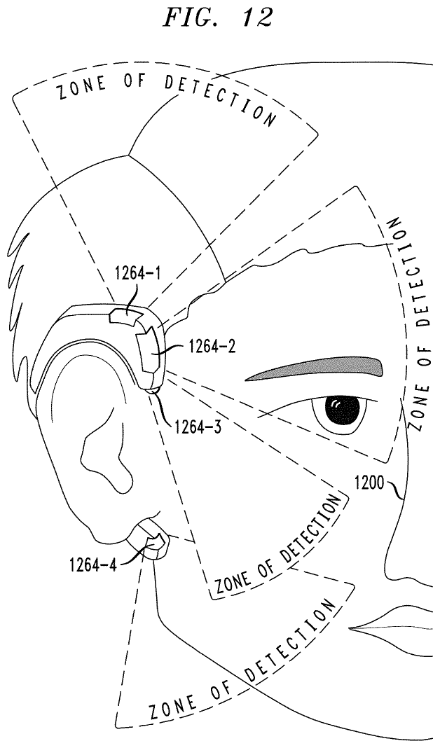

Additionally or alternatively, as detailed herein, one or more embodiments includes generating and/or implementing a mask device that encompasses only the face-touching detection and behavioral modification functionalities (i.e., such an embodiment does not include a filtration mechanism or goggles and/or protective eyewear). By way of example, FIG. 12 is a diagram illustrating a view of one or more components of a mask device (worn by user 1200) in an example embodiment. Specifically, FIG. 12 depicts an over-ear assembly portion of a mask device which includes IR sensors 1164-1, 1164-2, 1164-3 and 1164-4, as well as the sensors' corresponding zones of detection.

Further, in at least one embodiment, the device records all sensor values into a circular buffer or other memory storage device. If the completion of a sneeze is registered, a snapshot of all sensor data that proceeded the event is copied from the temporary buffer into long-term memory, and uploaded to the cloud as training data. In such an embodiment, prior to any long-term memory storage or upload or communication with any external device, the data from any sonic transducers is abstracted to a machine-readable description of momentary event states such that the sampling resolution is meaningfully descriptive of the respiratory characteristics but that no inadvertently recorded speech is audible or machine-decipherable. In one or more embodiments, a band cut filter or other signal processing is first applied to the sample prior to down-sampling in order to reduce the presence of otherwise decipherable vocal information.

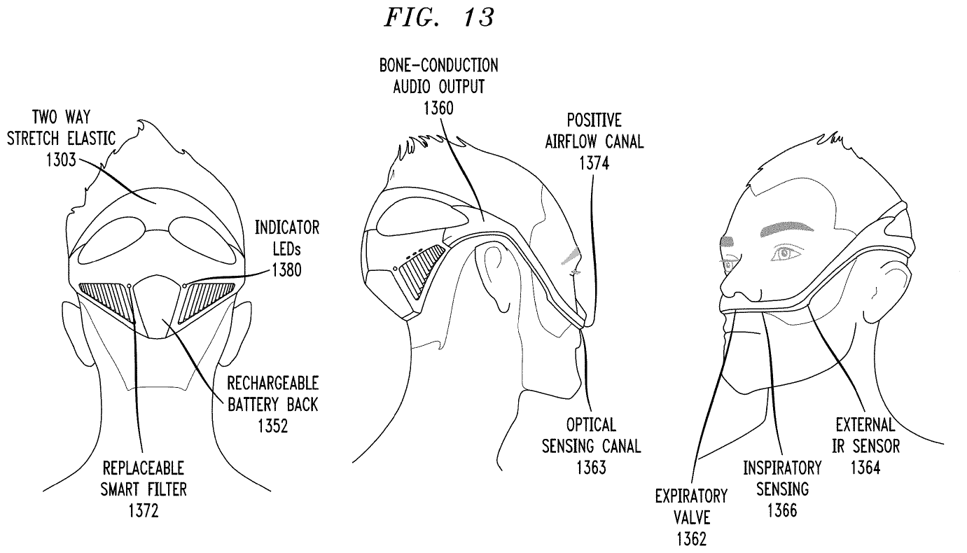

FIG. 13 is a diagram illustrating multiple views of one or more components of a mask device in an example embodiment. By way of illustration, FIG. 13 depicts a head assembly 1303 which includes two-way stretch elastic, indicator LEDs 1380, rechargeable battery pack 1352, and replaceable smart filter 1372. Additionally, FIG. 13 depicts bone-conducting audio output component 1360, positive airflow canal 1374, and optical sensing canal 1363. Further, FIG. 13 depicts expiratory valve 1362, inspiratory sensing component(s) 1366, and external IR sensor 1364.

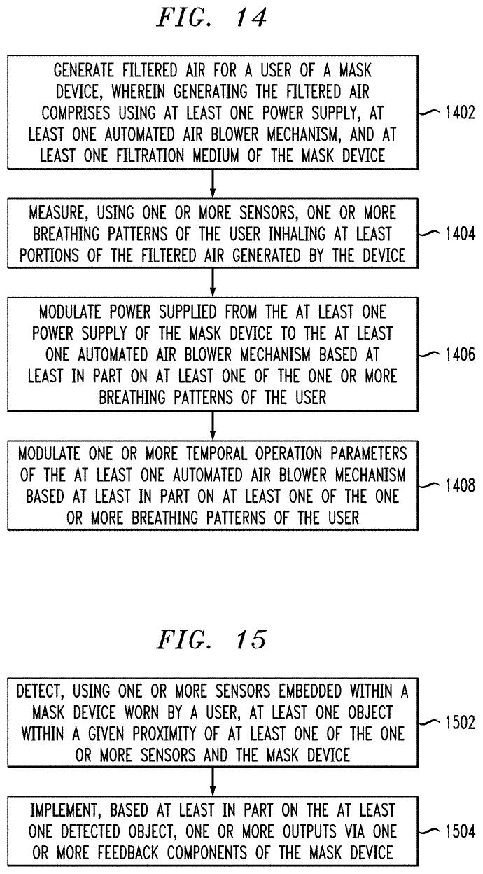

FIG. 14 is a flow diagram illustrating techniques according to an example embodiment. Step 1402 includes generating filtered air for a user of a mask device, wherein generating the filtered air comprises using at least one power supply, at least one automated air blower mechanism, and at least one filtration medium of the mask device.

Step 1404 includes measuring, using one or more sensors, one or more breathing patterns of the user inhaling at least portions of the filtered air generated by the device. In at least one embodiment, measuring, using the one or more sensors, one or more breathing patterns of the user includes measuring at least one respiration intensity pattern of the user and/or measuring at least one temporal respiratory pattern of the user. Additionally or alternatively, in one or more embodiments, measuring, using the one or more sensors, one or more breathing patterns of the user includes measuring one or more breathing patterns associated with each nostril of the user. In such an embodiment, measuring one or more breathing patterns associated with each nostril of the user includes measuring back-pressure differences associated with a first nostril of the user versus a second nostril of the user. Further, such an embodiment can include determining, based at least in part on the one or more breathing patterns associated with each nostril of the user, a frequency of variability associated with the user's alternating inspiratory nostril breaching.

As such, in accordance with one or more embodiments, the mask device can measure, via the sealed nasal cannula, the natural amplitude, intensity, and/or patterns of a user's changing nostril inhaling. Based on such measurements, such an embodiment can include directing air to one nostril or another, and detecting back-pressure differences from one nostril versus another. Using that information, such an embodiment can also include deducing a current dominant nostril, and determining related frequency and variability measures (i.e., the user's body's own alternating inspiratory nostril breathing). Such a determination can be used, for example, as a biomarker (e.g., such values can be compared to known benchmarks (e.g., healthy inspiratory systems, systems with respiratory conditions, etc.)).

Step 1406 includes modulating power supplied from the at least one power supply of the mask device to the at least one automated air blower mechanism based at least in part on at least one of the one or more breathing patterns of the user. In at least one embodiment, modulating the power supplied from the at least one power supply to the at least one automated air blower mechanism is based at least in part on a respiration intensity pattern of the user.

Step 1408 includes modulating one or more temporal operation parameters of the at least one automated air blower mechanism based at least in part on at least one of the one or more breathing patterns of the user. In one or more embodiments, modulating the one or more temporal operation parameters of the at least one automated air blower mechanism includes synchronizing output from the at least one automated air blower mechanism to a temporal respiratory pattern of the user. In such an embodiment, synchronizing output from the at least one automated air blower mechanism to a temporal respiratory pattern of the user includes incorporating a determined amount of latency attributed to one or more portions of the device.

FIG. 15 is a flow diagram illustrating techniques according to an example embodiment. Step 1502 includes detecting, using one or more sensors embedded within a mask device worn by a user, at least one object within a given proximity of at least one of the one or more sensors and the mask device.

Step 1504 includes implementing, based at least in part on the at least one detected object, one or more outputs via one or more feedback components of the mask device. In at least one embodiment, implementing one or more outputs includes generating an audio output, using at least one bone transducer, in response to detecting the at least one object, generating electrical stimulation, using one or more electrodes, in response to detecting the at least one object, and/or generating, using one or more haptic feedback components, a vibration in response to detecting the at least one object.

The techniques depicted in FIG. 15 can also include detecting, using at least a portion of the one or more sensors embedded within the mask device, mouth-breathing performed by the user, and implementing, based at least in part on the detected mouth-breathing, one or more outputs via one or more feedback components.

In at least one embodiment, an example device includes at least one power supply, at least one automated air blower mechanism coupled to the at least one power supply, at least one filtration medium, and at least one sealed nasal cannula with one or more air ports. The example devices also includes one or more sensors configured to measure one or more breathing patterns of a user of the device, and a set of one or more channels connecting the at least one automated air blower mechanism to the at least one filtration medium and the at least one sealed nasal cannula.

By way of example, in one or more embodiments, measuring the volume of air inhaled by a user can involve and/or include multiple different sensing approaches. For instance, at least one atmospheric pressure sensor (e.g., pressure sensor(s) 870 depicted in FIG. 8) allows the mask device to compute the density of the air outside of the device, as well as the relative concentration of oxygen (without having to measure the concentration of oxygen directly). To determine the intensity of a user's inspiratory breath, at least one embodiment includes measuring fluctuations in the pressure of the one or more inspiratory breath channels (e.g., channel 609 depicted in FIG. 6). The more the pressure is reduced (e.g., via suction), the harder the user is breathing. In an example embodiment, the mask device increases power to the automated air blower mechanism (e.g., blower mechanism 872 in FIG. 8) to increase the pressure to a level above the outside atmosphere. A lookup table within software can enable the device to determine at least one relationship between the amount of power supplied to the blower necessary to increase the pressure to this appropriate level, and the amount of air being inhaled by the user.

Also, in at least one embodiment, the set of one or more channels enables delivery of air, output by the at least one automated air blower mechanism and filtered by the at least one filtration medium, to the at least one sealed nasal cannula. Further, the example device includes a memory configured to store program instructions, and a processor operatively coupled to the memory to execute the program instructions to modulate power supplied from the at least one power supply to the at least one automated air blower mechanism based at least in part on at least one of the one or more breathing patterns of the user, and modulate one or more temporal operation parameters of the at least one automated air blower mechanism based at least in part on at least one of the one or more breathing patterns of the user.

In one or more embodiments, modulating power can increase or decrease the power supply supplied to the air blower mechanism. For example, if the user is inhaling more intensely and/or strongly, then less power made be required to be supplied to the air blower mechanism. For the temporal operation parameters (e.g., timing), when the user is about to take a breath, the power supply to the air blower mechanism temporarily increases, and then decreases and/or temporarily terminates when the user is expected to be exhaling, thereby making efficient use of the power.

Additionally, in at least one embodiment, modulating the power supplied from the at least one power supply to the at least one automated air blower mechanism is based at least in part on a respiration intensity pattern of the user. Further, modulating the one or more temporal operation parameters of the at least one automated air blower mechanism can include synchronizing output from the at least one automated air blower mechanism to a temporal respiratory pattern of the user. In such an embodiment, synchronizing output from the at least one automated air blower mechanism to a temporal respiratory pattern of the user includes incorporating a determined amount of latency attributed to one or more portions of the device.

Also, in one or more embodiments, the one or more sensors include one or more distance sensors, one or more motion sensors, one or more infrared sensors, and/or one or more pressure sensors. Additionally or alternatively, at least one embodiment includes one or more feedback components, wherein the processor is operatively coupled to the memory to execute the program instructions to detect, using at least a portion of the one or more sensors, at least one object within a given proximity of the device, and implement, based at least in part on the at least one detected object, one or more outputs via at least one of the one or more feedback components. In such an embodiment, the one or more feedback components can include one or more electrodes, one or more bone transducers, and/or one or more haptic feedback components. Further, one or more such embodiments can include at least one protective eyewear component.

Also, in one or more embodiments, the main body of such a mask device can be manufactured and/or molded out of a single piece of material (e.g., silicon). In such an embodiment, the first set of one or more channels and/or the second set of one or more channels can include portions of the single molded device comprising internal channels and/or features for air movement.

Such sensors can include, for example, one or more distance sensors, one or more motion sensors, one or more infrared sensors, and/or one or more pressure sensors. Additionally, such feedback components can include, for example, a haptic (e.g., vibration) component, one or more electrodes (e.g., one or more TENS electrodes) and/or one or more bone transducers (also referred to herein as bone conductor transducers).

In at least one embodiment, the at least one power supply of such an example device as detailed above can include at least one rechargeable battery, and the at least one filtration medium can include one or more particulate filter discs. Additionally or alternatively, such an example device can include at least one protective eyewear component.

The techniques depicted in FIG. 14 and/or FIG. 15 can also, as described herein, include providing a system, wherein the system includes distinct software modules, each of the distinct software modules being embodied on a tangible computer-readable recordable storage medium. All such modules (or any subset thereof) can be on the same medium, or each can be on a different medium, for example. The modules can include any or a combination of multiple of the components shown in the figures and/or described herein. In an example embodiment, the modules can run, for example, on a hardware processor. The method steps can then be carried out using the distinct software modules of the system, as described above, executing on a hardware processor. Further, a computer program product can include a tangible computer-readable recordable storage medium with code adapted to be executed to carry out at least one method step described herein, including the provision of the system with the distinct software modules.

Additionally, the techniques depicted in FIG. 14 and/or FIG. 15 can be implemented via a computer program product that can include computer useable program code that is stored in a computer readable storage medium in a data processing system, and wherein the computer useable program code was downloaded over a network from a remote data processing system. Also, in an example embodiment, the computer program product can include computer useable program code that is stored in a computer readable storage medium in a server data processing system, and wherein the computer useable program code is downloaded over a network to a remote data processing system for use in a computer readable storage medium with the remote system.

An example embodiment or elements thereof can be implemented in the form of an apparatus including a memory and at least one processor that is coupled to the memory and configured to perform exemplary method steps.

Other techniques can be used in association with one or more embodiments. Accordingly, the particular processing operations and other functionality described herein are presented by way of illustrative example only, and should not be construed as limiting the scope of the invention in any way. For example, the ordering of the process steps may be varied in one or more other embodiments, or certain steps may be performed concurrently with one another rather than serially. Also, the process steps or subsets thereof may be repeated periodically in conjunction with respective distinct instances of use.

Aspects of the present invention are described herein with reference to flowchart illustrations and/or block diagrams of methods, apparatus (systems), and computer program products according to one or more embodiments. It will be understood that each block of the flowchart illustrations and/or block diagrams, and combinations of blocks in the flowchart illustrations and/or block diagrams, can be implemented by computer readable program instructions.

The flowchart and block diagrams in the Figures illustrate the architecture, functionality, and operation of possible implementations of systems, methods, and computer program products according to various embodiments. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Additionally, networks disclosed herein can be implemented, for example, using one or more processing platforms. Such a processing platform can include, by way of example, at least one processing device comprising a processor coupled to a memory.

In one or more embodiments, portions of a network as disclosed herein can illustratively include cloud infrastructure. The cloud infrastructure, in at least one such embodiment, can include a plurality of containers implemented using container host devices, and/or can include container-based virtualization infrastructure configured to implement Docker containers or other types of Linux containers.

The cloud infrastructure can additionally or alternatively include other types of virtualization infrastructure such as virtual machines implemented using a hypervisor. Additionally, the underlying physical machines include one or more distributed processing platforms that include one or more storage systems.

Such cloud infrastructure as described above can, by way of example, represent at least a portion of one processing platform. Another example of such a processing platform can include, for instance, a plurality of processing devices which communicate with one another over a network. As yet another processing platform example, portions of a given processing platform in one or more embodiments can include converged infrastructure.

The particular processing platforms described above are presented by way of example only, and a given network can include additional or alternative processing platforms, as well as numerous distinct processing platforms in any combination, with each such platform comprising one or more computers, servers, storage devices and/or other processing devices.

Further, in accordance with one or more embodiments, processing devices and other network components can communicate with one another using a variety of different communication protocols and associated communication media.

It should again be emphasized that one or more of the embodiments described herein are presented for purposes of illustration only. Many variations may be made in the particular arrangements shown. Moreover, the assumptions made herein in the context of describing one or more illustrative embodiments should not be construed as limitations or requirements of the invention, and need not apply in one or more other embodiments. Numerous other alternative embodiments within the scope of the appended claims will be readily apparent to those skilled in the art.

What is claimed is:

1. A device comprising:
at least one power supply;
at least one automated air blower mechanism coupled to the at least one power supply;
at least one filtration medium;
at least one sealed nasal cannula with one or more air ports;
one or more sensors configured to measure one or more breathing patterns of a user of the device;
a set of one or more channels connecting the at least one automated air blower mechanism to the at least one filtration medium and the at least one sealed nasal cannula;
a memory configured to store program instructions; and
a processor operatively coupled to the memory to execute the program instructions to:
modulate power supplied from the at least one power supply to the at least one automated air blower mechanism based at least in part on at least one of the one or more breathing patterns of the user; and
modulate one or more temporal operation parameters of the at least one automated air blower mechanism based at least in part on at least one of the one or more breathing patterns of the user.

2. The device of claim 1, wherein modulating the power supplied from the at least one power supply to the at least one automated air blower mechanism is based at least in part on a respiration intensity pattern of the user.

3. The device of claim 1, wherein modulating the one or more temporal operation parameters of the at least one automated air blower mechanism comprises synchronizing output from the at least one automated air blower mechanism to a temporal respiratory pattern of the user.

4. The device of claim 3, wherein synchronizing output from the at least one automated air blower mechanism to a temporal respiratory pattern of the user comprises incorporating a determined amount of latency attributed to one or more portions of the device.

5. The device of claim 1, wherein the one or more sensors comprise at least one of one or more distance sensors, one or more motion sensors, one or more infrared sensors, and one or more pressure sensors.

6. The device of claim 1, further comprising:
one or more feedback components;
wherein the processor is operatively coupled to the memory to execute the program instructions to:
detect, using at least a portion of the one or more sensors, at least one object within a given proximity of the device; and
implement, based at least in part on the at least one detected object, one or more outputs via at least one of the one or more feedback components.

7. The device of claim 6, wherein the one or more feedback components comprise at least one of one or more electrodes, one or more bone transducers, and one or more haptic feedback components.

8. The device of claim 1, further comprising:
at least one protective eyewear component.

9. A computer-implemented method comprising:
generating filtered air for a user of a mask device, wherein generating the filtered air comprises using at least one power supply, at least one automated air blower mechanism, and at least one filtration medium of the mask device;
measuring, using one or more sensors, one or more breathing patterns of the user inhaling at least portions of the filtered air generated by the device;
modulating power supplied from the at least one power supply of the mask device to the at least one automated air blower mechanism based at least in part on at least one of the one or more breathing patterns of the user; and
modulating one or more temporal operation parameters of the at least one automated air blower mechanism based at least in part on at least one of the one or more breathing patterns of the user;
wherein the method is carried out by at least one processor associated with the mask device.

10. The computer-implemented method of claim 9, wherein modulating the power supplied from the at least one power supply to the at least one automated air blower mechanism is based at least in part on a respiration intensity pattern of the user.

11. The computer-implemented method of claim 9, wherein modulating the one or more temporal operation parameters of the at least one automated air blower mechanism comprises synchronizing output from the at least one automated air blower mechanism to a temporal respiratory pattern of the user.

12. The computer-implemented method of claim 11, wherein synchronizing output from the at least one automated air blower mechanism to a temporal respiratory pattern of the user comprises incorporating a determined amount of latency attributed to one or more portions of the device.

13. The computer-implemented method of claim 9, wherein measuring, using the one or more sensors, one or more breathing patterns of the user comprises measuring at least one respiration intensity pattern of the user.

14. The computer-implemented method of claim 9, wherein measuring, using the one or more sensors, one or more breathing patterns of the user comprises measuring at least one temporal respiratory pattern of the user.

15. The computer-implemented method of claim 9, wherein measuring, using the one or more sensors, one or more breathing patterns of the user comprises measuring one or more breathing patterns associated with each nostril of the user.

16. The computer-implemented method of claim 15, wherein measuring one or more breathing patterns associated with each nostril of the user comprises measuring back-pressure differences associated with a first nostril of the user versus a second nostril of the user.

17. The computer-implemented method of claim 15, further comprising:
determining, based at least in part on the one or more breathing patterns associated with each nostril of the user, a frequency of variability associated with the user's alternating inspiratory nostril breaching.

18. A computer-implemented method comprising:
detecting, using one or more sensors embedded within a mask device worn by a user, at least one object within a given proximity of at least one of the one or more sensors and the mask device; and
implementing, based at least in part on the at least one detected object, one or more outputs via one or more feedback components of the mask device;
wherein the method is carried out by at least one processor associated with the mask device.

19. The computer-implemented method of claim 18, further comprising:
detecting, using at least a portion of the one or more sensors embedded within the mask device, mouth-breathing performed by the user; and
implementing, based at least in part on the detected mouth-breathing, one or more outputs via one or more feedback components.

20. The computer-implemented method of claim 18, wherein implementing one or more outputs comprises at least one of generating an audio output, using at least one bone transducer, in response to detecting the at least one object, generating electrical stimulation, using one or more electrodes, in response to detecting the at least one object, and generating, using one or more haptic feedback components, a vibration in response to detecting the at least one object.
